(19) United States
(12) Patent Application Publication
(10) Pub. No.: US 2009/0155830 A1
(43) Pub. Date: Jun. 18, 2009

(54) Composition and a kit for detecting early apoptosis in frozen umbilical cord and a method therefor

(75) Inventor: Il-Hoan Oh, Seoul (KR)

Correspondence Address: JHK LAW, P.O. BOX 1078, LA CANADA, CA, 91012-1078, US

(73) Assignee: Catholic University Industry Academic Cooperation Foundation, Seoul (KR)

(21) Appl. No.: 11/757,317

(22) Filed: Jun. 1, 2007

(30) Foreign Application Priority Data
Jun. 1, 2007 (KR) 10-2006-0049607

(51) Int. Cl. C12Q 1/02

(52) U.S. Cl. 435/29

(57) ABSTRACT

There are provided a composition and kit for detecting early apoptosis in cryopreserved umbilical cord blood stem cells, and a method therefor. According to the present invention, when the umbilical cord blood stem cells are cryopreserved and later used for cell therapy, the quality of umbilical cord blood is assessed and early apoptosis in the umbilical cord blood stem cells is detected. The obtained resulting data, which can be used as a quality reference for umbilical cord blood required for transplantation, reflect the engraftment levels after in vivo transplantation of the stem cells, and thus allow prediction of the engraftment levels from the results.

### BACKGROUND OF THE INVENTION

1. Field of the Invention

The present invention relates to a composition and a kit for detecting early apoptosis in cryopreserved umbilical cord blood stem cells, and a method therefor.

2. Description of the Related Art

Umbilical cord blood (UCB) has been increasingly used throughout the world as a source of cryopreserved stem cells, and this raises issues regarding the importance of UCB quality control.

Cryopreserved umbilical cord blood is a source of the stem cells that are critical for treating a variety of incurable diseases in adults or infants (Barker and Wagner 2003, Fernandez, et al 1999, Gluckman, et al 2001). It has been known that matching of human leukocyte antigens (HLAs) for the umbilical cord blood transplantation, and the number of cells are important factors for determining post-transplantation prognosis (Gluckman, et al 2001, Laughlin, et al 2001, Laughlin, et al 2004, Rocha, et al 2000, and Rubinstein, et al 1998).

Particularly, due to the use of a small number of cells, various side effects may occur during the transplantation, such as delayed engraftment. As a result, there have been attempts to overcome such limitations by using in vitro culture, but that too has limitations (Jaroscak, et al 2003). In order to solve the above problems, there have been attempts to develop mixed engraftment (Barker, et al 2001, and Kim, et al 2004). Among such attempts, it has been reported that the number of viable cells may be more critical than a sufficiently large number of the cells (Laughlin, et al 2001, Laughlin, et al 2004, and Rocha, et al 2004). Also, it has been reported that the number of CD34 positive cells in the monocytes (Baum, et al 1992, and Larochelle, et al 1996) is more significant than the total counts of monocytes (Gluckman, et al 2001, and Rocha, et al 2004) in the umbilical cord blood transplantation because the total counts of CD34 positive cells represent the actual number of the stem cells, and thus more useful in overcoming the immune rejection of transplanted stem cells (Wagner, et al 2002, and Yap, et al 2000). By way of a similar example, it has been reported that the number of CD34+ cells can be used to facilitate the prediction of the later-occurring engraftment levels of the umbilical cord blood in 562 cases of the umbilical cord blood transplantations (Aroviita, et al 2003).

In spite of the above suggestions, some studies have reported that the CD34 positive cells are often shown to be positive in the common detection of apoptosis using 7-minoactinomycin D (7-AAD) or propidium iodide (PI) staining (de Boer, et al 2002, Mastino, et al 2003, and Schuurhuis, et al 2001), which leads to undesirable clinical results (Allan, et al 2002, and de Boer, et al 2002).

Accordingly, the present inventors have conducted extensive studies and found that:

(1) in order to measure early apoptosis, which could not be detected using conventional methods, an Annexin-V method was used to confirm the possibility that apoptosis of the cryopreserved umbilical cord blood cells, which have been previously thought to be viable cells, are actually scheduled;

(2) the stem cells which have undergone early apoptosis had substantially no engraftment ability after they were transplanted into NOD/SCID mice; and

(3) the resulting data from the Annexin-V method can be used as a marker for quality control in order to predict the course after transplantation of the cryopreserved umbilical cord blood.

The present inventors have also found that the early apoptosis in the CD34 positive cells can be one of the factors that may cause variation in the quality of the cryopreserved umbilical cord blood. Accordingly, they have measured the proportion of the cells that were shown to be positive for Annexin-V staining capable of detecting early apoptosis in the cells that were shown to be positive for CD34, as well as to be negative for general apoptosis, as in propidium iodide (PI) staining.

As a result of the studies, it has been found that the proportion of the cells which undergone early apoptosis in the cells from the cryopreserved umbilical cord blood is in a wide range of diversity, and the cells from the umbilical cord blood that has been cryopreserved for 5 to 7 years exhibit a wider range of diversity and a higher level of early apoptosis. Also, it has been confirmed that when a bone marrow regeneration ability is observed after the cell groups showing the early apoptosis have been transplanted into NOD/SCID mice, most of the regeneration ability of the umbilical cord blood was observed in the Annexin-V negative group, which can be then used as an indicator of regeneration ability of the umbilical cord blood. Moreover, by comparing the staining levels of Annexin-V, and the engraftment levels of the umbilical cord blood after transplantation with each other through the analysis of two discrete cohort groups, it has been found that the staining levels and the engraftment levels have a significant relationship with each other. Accordingly, it has also been found that the resulting data from such the measurement method can be a critical marker for selecting cryopreserved umbilical cord blood before transplantation of the umbilical cord blood, as well as could prevent a low level of engraftment caused from the quality problems of the umbilical cord blood.

### SUMMARY OF THE INVENTION

It is an object of the present invention to provide a method for the quality control of umbilical cord blood when the umbilical cord blood stem cells are cryopreserved, and later used for cell therapy.

It is another object of the present invention to provide a method for detecting early apoptosis in umbilical cord blood stem cells, and then using the obtained resulting data as a quality reference of umbilical cord blood when the umbilical cord blood required for transplantation is selected and screened in a cord blood bank.

### BRIEF DESCRIPTION OF THE DRAWINGS

FIG. 1 is a diagram showing various Annexin-V positivities in the umbilical cord blood CD34+PI(−) cells, in which FIG. 1A is a representative diagram of early apoptosis as detected in the CD34+PI(−) cells, in which umbilical cord blood monocytes were stained with Annexin V-FITC, CD34-APC and PI, an isotype antibody was used as a reference, and the amount (%) of Annexin V(+) cells in the CD34+PI(−) cells was calculated; FIG. 1B is a diagram for the measurement as described above, using freshly collected peripheral blood cells; FIG. 1C is a diagram showing the rate of early apoptosis occurring in cryopreserved umbilical cord blood, as analyzed using an Annexin V-binding reaction, in which the early apoptosis was analyzed by using both of erythrocyte fractionation (black) or Ficoll-Hypaque isolation (blank), and the horizontal bar represents a mean value; and FIG. 1D is a diagram showing the levels of the early apoptosis occurring in freshly collected umbilical cord blood at various collecting times, as represented with Annexin V(+) cell groups, in which the Annexin-V positivities of CD34+PI(−) cells (black), or the PI-negative total monocytes (white) are shown.

FIG. 2 is a diagram for analysis of the physiological meanings of the Annexin-V-binding reaction in the umbilical cord blood, in which FIG. 2A is a diagram showing the isolation of cells which were shown to be respectively strongly positive, weakly positive, and negative to Annexin V in the CD34+PI(−) cells among the CD34+ cells, using a flow cytometer; FIG. 2B is a diagram showing the engraftment levels of human-derived cells, as measured using a human-specific CD45/71 antibody at 8 weeks after transplantation of the same counts (3×10⁴) of the CD34+PI(−)¹ cells into NOD/SCID mice (Mean value is expressed as SEM (n=5)); FIG. 2C is a diagram showing the separation of the CD34+PI(−)/Annexin V(−) cells from cryopreserved umbilical cord blood (into Fr1 and Fr2) according to their Annexin-V negativities; and FIG. 2D is a diagram showing the engraftment levels of human-derived cells, as measured after transplantation of the cells into NOD/SCID mice, and the differentiation pattern of each cell, as analyzed using human-specific lymphatic (CD19/20) and myeloid (CD13/15) antibodies.

FIG. 3 is a diagram for analysis of the engraftment predictability after transplantation of umbilical cord blood, as measured using the Annexin-V method, in which the CD34+PI(−) cells from the umbilical cord blood, which were randomly extracted from the umbilical cord blood that had been stored for 5 to 7 years, were measured for their Annexin-V negativities, and the total monocytes were transplanted into NOD/SCID mice (3×10⁴ CD34+PI(−) cells), so as to measure the engraftment levels of the cells at 8 weeks after the transplantation. Here, the results of follow-up studies of two discrete groups are each shown in the left and right panels, and the horizontal bars represent a mean value (in which r² is a correlation coefficient).

### DESCRIPTION OF THE PREFERRED EMBODIMENTS

In one preferred embodiment, the present invention provides a method for the quality control of umbilical cord blood when the umbilical cord blood stem cells are cryopreserved, and used later for the purpose of cell therapy.

According to the present invention, it is found that the early apoptosis occurs widely in umbilical cord blood stem cells, wherein the early apoptosis could not be detected with the cell viability test using conventional methods (a trypan blue dye exclusion assay, a propidium iodide staining method, a 7-AAD staining method, etc.).

In another embodiment, the present invention provides a method for detecting early apoptosis using an Annexin V staining method, which is one of the methods for detecting membrane flip-flop of the cell membrane containing phosphatidyl serine groups, in which the results of the measurement reflect the engraftment levels after in vivo transplantation of the stem cells; and then predicting the engraftment level from the results of the above measurement.

In still another embodiment, the present invention provides a method for detecting early apoptosis in umbilical cord blood stem cells, and then using the obtained resulting data as a quality reference of umbilical cord blood when the umbilical cord blood required for transplantation is selected and screened in a cord blood bank.

Further, as proved in the present invention, since various levels of early apoptosis are observed in cryopreserved umbilical cord blood, and a significant level of early apoptosis is also observed even in freshly collected umbilical cord blood, the level of early apoptosis in umbilical cord blood collected prior to a freezing step is likely to vary widely, and thus the early apoptosis occurring in the umbilical cord blood can be detected before the step of cryopreserving the umbilical cord blood collected from an umbilical cord blood bank, etc., as well as immediately before transplanting the umbilical cord blood after thawing the frozen umbilical cord blood.

In a first embodiment, the present invention provides a database for the marked Annexin-V positivities in umbilical cord blood in a fresh or cryopreserved state, or an umbilical cord blood product group having the marked Annexin-V positivities in the CD34 positive cell groups.

In a second embodiment, the present invention provides a kit which is designed to check the level of early apoptosis by means of flow cytometry, using some of the test samples contained in a supplementary bag that has been recently used to assess the quality of umbilical cord blood immediately before transplantation, and then transplant the umbilical cord blood.

In a third embodiment, the present invention provides a kit for measuring the CD34 positivity and the membrane flip-flop of phosphatidyl serine groups at the same time. The kit includes a complex kit in the various forms for measuring Annexin-V using flow cytometry, in addition to PI or 7-ADD for measuring CD34 antibodies and general apoptosis.

### EXAMPLES

Hereinbelow, the present invention will be described with reference to Examples. However, the following Examples are provided only for the purpose of illustrating the present invention, and the scope of the present invention is not intended to be limited to Examples.

### Methods

### Cell Isolation

Monocytes were isolated from cryopreserved or fresh umbilical cord blood using a Ficoll-Hypaque (Amersham Bioscience, Uppsala, Sweden) density gradient centrifugation (<1.077), and then the cells were suspended in a Ca⁺⁺/Mg⁺⁺-free HF2 solution (containing a Hank's balanced salt solution with 2% bovine serum). The CD34 positive cells were isolated using a positive isolation kit (DynalBiotech, Oslo, Norway).

### Annexin V Binding Reaction and Flow Cytometric Analysis

Thawed or fresh umbilical cord blood cells were bound to an anti-CD34-APC (BD Pharmingen, San Diego, Calif., USA) antibody, and then washed. The resultant umbilical cord blood cells were bound with Annexin V-FITC (BD Pharmingen) in a binding solution for 30 minutes, and then stained with Propidium Iodide or 7-AAD, thereby detecting apoptosis. Then, the cells shown to be positive were analyzed using a flow cytometer (FacsCaliber, BD Bioscience, San Jose, Calif., USA).

Test on Transplantation of Umbilical Cord Blood Cells into NOD/SCID Mice

An experiment with xenotransplantation of umbilical cord blood cells into NOD/SCID (nonobese diabetic (NOD)/severe combined immunodeficiency (SCID), Shultz, et al 1995) mice was carried out in the same manner as the procedure in the prior art (Kim, et al 2004). In brief, the isolated CD34+ cells or the total monocytes were transplanted into mice exposed to 300 cGy radiation, and then 100 mg/L of ciprofloxacin (Bayer AG, Leverkusen, Germany) was administered to the mice for 3 weeks. In order to measure the engraftment level in these mice, antibody response tests were carried out using an antihuman CD45-PE antibody (BD Pharmingen), an antihuman CD71-PE antibody (BD Pharmingen), an antihuman CD19, 20 antibody (BD Pharmingen) and antihuman CD13, 15 antibodies (BD Pharmingen). At this time, 5% human serum and a 2.4G2 antibody (an antimouse Fc receptor antibody) were used to avoid non-specific reactions.

### Statistical Analysis

All of the results were presented as mean values±SEMs, and verified through a significant Student's t-test and a Pearson correlation coefficient.

### Results

It was found that various levels of early apoptosis are observed in the cryopreserved umbilical cord blood cells.

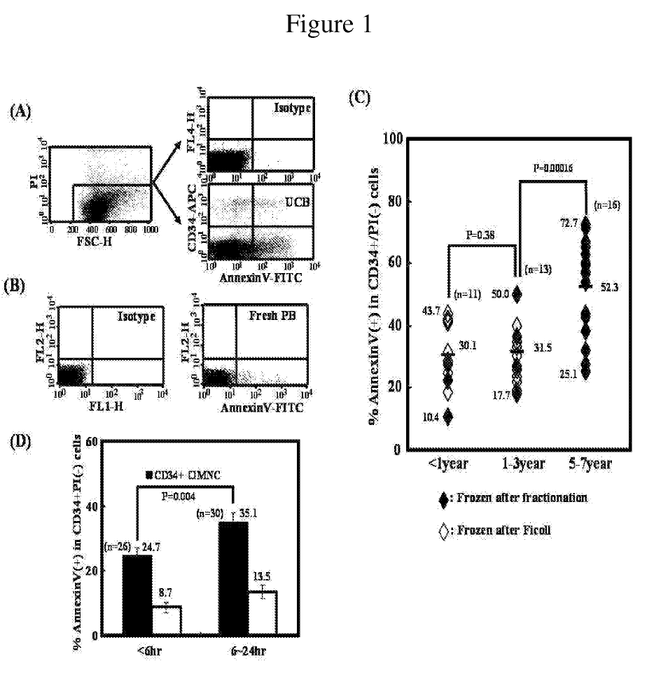

In the present invention, it was analyzed whether the cells shown to be CD34-positive, PI-negative and Annexin V-positive are present in the cryopreserved umbilical cord blood cells (FIG. 1A), in order to confirm whether early apoptosis in the cryopreserved umbilical cord blood may be used as a reference for quality control. The Annexin-V binding reaction was used as one of the methods for detecting early apoptosis on the basis of a principle that phosphatidyl serine present inside the cell membrane is detected through the membrane flip-flop (Martin, et al 1995, Vermes, et al 1995). Specificity/Accuracy of the measurement method according to the present invention were re-confirmed from the fact that the Annexin-V-positive reaction occur at a level of 2% or less in freshly collected peripheral blood (FIG. 1B).

The CD34+PI(−) cells, which was formerly thought to be viable stem cells from umbilical cord blood that had been cryopreserved for a period of 1 year or less, were analyzed through the above method. As a result, it was revealed that there exists difference in the positivities between the populations in a wide range from 10 to 44% (mean 30±11%) (FIG. 1C). Also, it was revealed that the similar results were obtained from the 13 umbilical cord bloods which had been cryopreserved for 1 to 3 years (32±11%, p=0.38), and there was no difference in the positivities between the populations that had been stored in an umbilical cord blood freezing bag and a cryovial (FIG. 1C). However, it was seen that there are significant differences in early apoptosis of the 16 umbilical cord bloods that had been stored for 5 to 7 years, and the umbilical cord bloods had higher mean values in early apoptosis (mean: 52±15%) (FIG. 1C).

56 fresh umbilical cord bloods were tested to determine whether early apoptosis occurs only in the cryopreserved cells. As a result, it was surprisingly revealed that early apoptosis occurs to a similar extent (FIG. 1D). Unexpectedly, early apoptosis of the fresh umbilical cord bloods varied depending on the collection time of the umbilical cord blood, and the apoptosis was observed at a lower level in the cell group which had been collected within 6 hours after the parturition than in the umbilical cord blood which had been collected at 6 to 24 hours after the parturition (25% vs. 35%, P=0.004) (FIG. 1C). Accordingly, these results confirmed that early apoptosis can be detected early in the freshly obtained umbilical cord blood, as well as in the cryopreserved umbilical cord blood.

Functional meanings of Early Apoptosis on Stem Cell Transplantation

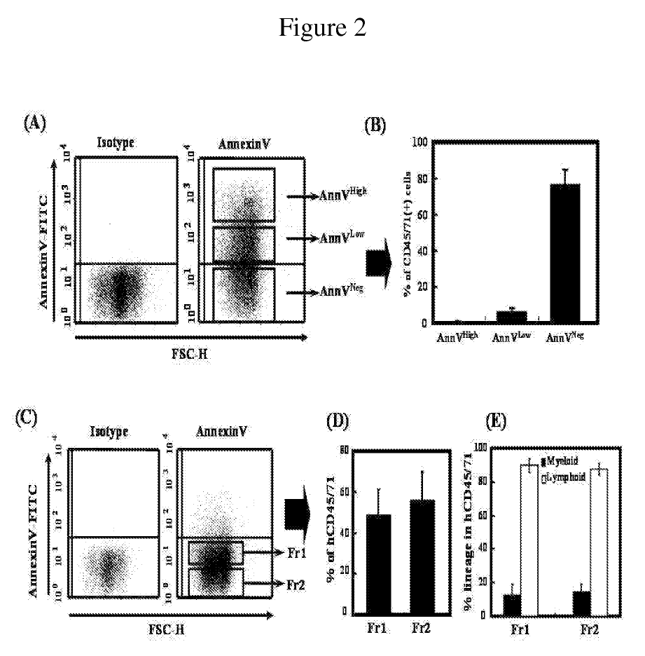

In the present invention, in order to determine whether the difference in the early apoptosis between the umbilical cord bloods as shown in the Annexin-V binding reaction is associated with the clinically important, post-transplantational engraftment ability, it was tested whether the Annexin-V negative cells may represent one cell group having an engraftment ability in one umbilical cord blood (FIG. 2A). As shown in FIG. 2A, the difference in the engraftment levels was tracked by isolating the cells that are negatively and positively bound to the Annexin V, respectively, among the cell groups shown to be CD34+PI(−), using a flow cytometer, followed by transplanting each of the cells into NOD/SCID mice (FIG. 2A). As a result, most of the Annexin-V(−) cells showed engraftment ability (70±9%, n=5; FIG. 2B), whereas the cell groups belonging to Annexin V^{low} or Annexin V^{high} show substantially no engraftment ability (6±3% and 0.5±0.4%; P=0.0006 and 0.0004). Also, there was not additional difference in the engraftment ability which results from the difference in the Annexin-V negativity in the Annexin V(−) cell groups, and there was no difference in the engraftment ability even if the cells were isolated respectively (FIG. 2C) and transplanted into the NOD/SCID mice(FIG. 2D), and there was no difference in the lymphatic and myeloid differentiations (FIG. 2E). Accordingly, the above results confirmed that the Annexin-V binding level in the CD34 positive cell groups can be a reference that can reflect the ability of the stem cells from one umbilical cord blood, that is, substantially most of the post-transplantational engraftment ability of the stem cells.

### Prediction Test on Post-transplantational Engraftment Level Through Pre-Screening of Annexin V

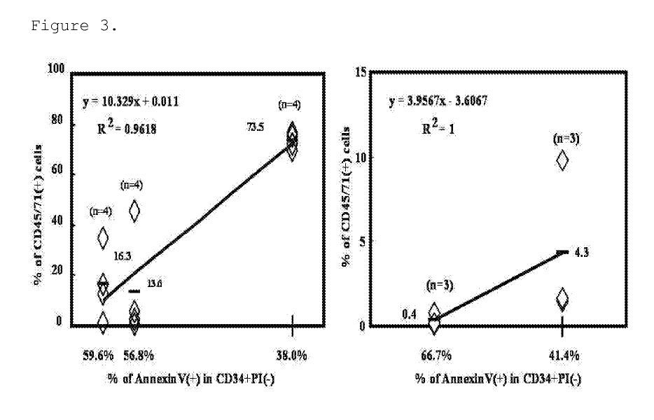

In order to determine whether the Annexin V binding level can be used to predict the engraftment level after the transplantation of the umbilical cord blood stem cells, randomly extracted umbilical cord bloods were screened, and then subjected to follow-up studies of two discrete groups to compare the post-transplantational engraftment levels from each other (left panel of FIG. 3). First, it was revealed that when three umbilical cord bloods which were shown to have Annexin-V positivities of 59%, 57% and 38%, respectively, among the CD34+PI(−) cells, were transplanted into the NOD/SCID mice, the engraftment levels in the NOD/SCID mice were 16%, 14% and 74%, respectively, indicating that the engraftment levels have a significant correlation with early apoptosis (r²=0.9618). Even in another follow-up study, it was revealed that the engraftment levels have a reverse correlation with early apoptosis when the umbilical cord bloods showing Annexin-V positivities of 67% and 41% were transplanted into the NOD/SCID mice, respectively (right panel of FIG. 3). The above results confirmed that an engraftment reaction can be predicted, as will be shown when the same umbilical cord blood will be transplanted later, by previously screening the Annexin V binding reaction for detecting the early apoptosis in the umbilical cord blood, and the results obtained by previously screening the umbilical cord blood to be transplanted can be used as a marker for obtaining a best transplantation result.

### Effects of the Invention

According to the present invention, when the umbilical cord blood stem cells are cryopreserved and later used for cell therapy, the quality of umbilical cord blood is assessed and early apoptosis in the umbilical cord blood stem cells is detected. The obtained results, which can be used as a quality reference for umbilical cord blood required for transplantation, reflect the engraftment levels after in vivo transplantation of the stem cells, and thus allow prediction of the engraftment levels from the results.

What is claimed is:

1. A method for detecting early apoptosis in umbilical cord blood cells by detecting the cell membrane flip-flop of phosphatidyl serine in the umbilical cord blood cells.

2. A kit for detecting early apoptosis in umbilical cord blood cells, comprising a substance for detecting the cell membrane flip-flop of phosphatidyl serine.

3. A method for screening an umbilical cord blood cell composition for transplantation, comprising a step of screening the cells that are CD34-positive and do not undergo the cell membrane flip-flop of phosphatidyl serine.
